(19) United States
(12) Patent Application Publication
(10) Pub. No.: US 2020/0060867 A1
(43) Pub. Date: Feb. 27, 2020

(54) Bi-Directional Prophylactic with Band

(71) Applicant: Zachary Guilford Shaffer, Shelby, MI (US)

(72) Inventor: Zachary Guilford Shaffer, Shelby, MI (US)

(21) Appl. No.: 15/798,757

(22) Filed: Oct. 31, 2017

(51) Int. Cl. A61F 6/04

(52) U.S. Cl. CPC A61F 6/04; A61F 2006/047

(57) ABSTRACT

Synergy: Band incorporated to pleated condom enables condom to be retained in the band for Bi-Directional use. Other applications could be possible. The design could improve global correct usage of condoms.

### TECHNOLOGY

Ive invented a Foolproof Sheath worn on the penis.

### Process

New process, Using pleats in manufacturing process enables sheath to be worn in either direction, Bi_Directional. The pleats enable sheath to be compressed and stored, accepted into the band. Pleats enable sheath to deploy Bi_Directionally from band, Not rolled.

### Useful

The bi_directional application prevents the condom from being worn improperly or backwards. This design lessens the unnecessary stress or strain on material. The incorporation of my technology will help prevent accidental pregnancies and certain types of sexually transmitted diseases, Because ease of application and foolproof design. No misuse caused by inside out or backwards application.

### Novel

The introduction of a Band to a prophylactic is new. The band can help restrict blood which enables the penis to retain a rigid or firmer state for a longer period of time.

### Nonobvious:

My invention is not using the industry standard of rolling the condom one direction to be worn on the penis.

### How to Make:

Injection mold technology in making the standard condom. The mold is modified to incorporate pleats and retaing band to produce the Bi-Directional Condom with Band.

Reason why;

Now their is no excuse not to wear a condom, Bi-Directional means no more frustration from applying condom backwards or wrong direction.

Pleats incorporated for compressing sheath to be accepted in to the band. Latex or latex free alergin free material. Band diploys length of prophylactic in either direction, Bi-Directional. Band either folds outward or is expanded to accept and store sheath, in manufacture process. Band has different density, firm, rigid than sheath to create stability and form. Also constricts penis to limit or restrict flow of blood exiting penis.

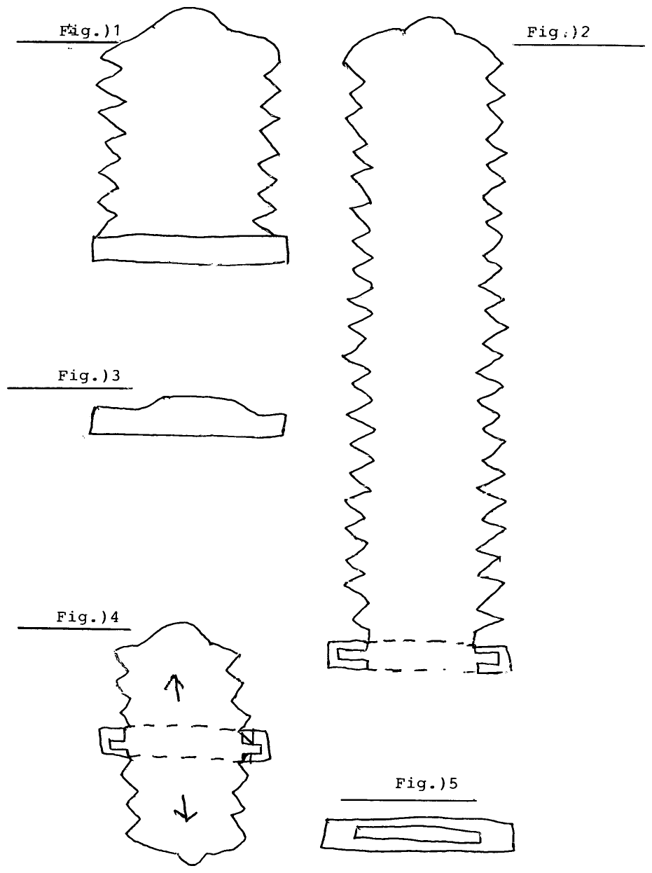

FIG. 1. ONe dimension side view semi deployed pleated sheath with band.

FIG. 2 One dimension side view fully deployed pleated sheath with retaining band.

FIG. 3 One dimension side view retaining band, Sheath can be deployed either direction.

FIG. 4 One dimension side view of sheath being deployed from retaining band in either direction.

FIG. 5 One dimension cutaway of retaining band.

substitute specification, this substitute specification contains no new matter.

the sheath and band is created by either dipping into a trough or injecting material into a mold.

the opening of the sheath is attached to the cavity surround inside the band. FIG. 2

folds or pleats incorporated into the sheath enable the sheath to be compressed and inserted into the hollow cavity of the retaing band. FIGS. 2,1,3.

the band cavity covers and stores the sheath until use. FIG. 5

the folds or pleats enable an inversion of the sheath if needed. the outside of the sheath can become the inside. (reversal of normal order). FIGS. 3,4.

the sheath is able to exit bi-directionally, while still being attached to the surround inside the band. FIG. 4.

1. Sheath is stored in the retaining band. Sheath is able to deploy in either direction. Material can be folded, pleated, or injected into the retaining band.
